(19) United States
(12) Patent Application Publication
(10) Pub. No.: US 2014/0271334 A1
(43) Pub. Date: Sep. 18, 2014

(54) BIODEGRADABLE MG BASED ALLOY AND IMPLANT

(71) Applicant: Korea Institute of Science and Technology, Seoul (KR)

(72) Inventors: Yu Chan KIM, Gyeonggi-do (KR); Hyun Kwang SEOK, Seoul (KR); Do Hyang KIM, Seoul (KR); Jeong Kyun KIM, Incheon (KR)

(73) Assignee: Korea Institute of Science and Technology, Seoul (KR)

(21) Appl. No.: 14/053,017

(22) Filed: Oct. 14, 2013

(30) Foreign Application Priority Data
Mar. 14, 2013 (KR) 10-2013-0027480

(51) Int. Cl. A61L 31/02; C22C 23/04

(52) U.S. Cl. CPC A61L 31/022; C22C 23/04
USPC 420/411

(57) ABSTRACT

A biodegradable Mg-based alloy and implant are provided. The biodegradable Mg-based alloy is represented with a composition equation Mg_{100-a-b-c}ZnₐLi_{b}Zr_{c}, wherein a, b, and c of the composition equation are wt % of Zn, Li, and Zr, respectively, and satisfy 0<a≦5, 1≦b≦3, and 0≦c≦1, respectively.

### CROSS-REFERENCE TO RELATED APPLICATION

This application claims priority to and the benefit of Korean Patent Application No. 10-2013-0027480 filed in the Korean Intellectual Property Office on Mar. 14, 2013, the entire contents of which are incorporated herein by reference.

### BACKGROUND OF THE INVENTION

(a) Field of the Invention

The present invention relates to a biodegradable Mg-based alloy and implant. More particularly, the present invention relates to a biodegradable Mg-based alloy and implant that can easily control a bio-decomposition speed and that have excellent strength and elongation.

(b) Description of the Related Art

Nowadays, a typical material of an implant that is used for a medical treatment is a metal material having an excellent mechanical property and workability. However, in spite of an excellent property of a metal, a metal implant has several problems such as stress shielding, image degradation, and implant migration.

In order to overcome a drawback of such a metal implant, research and development of a biodegradable implant were started. Research into medical applications of a biodegradable material was started through a polymer such as polylactic acid (PLA), polyglycolic acid (PGA), or PLGA, which is a copolymer thereof. However, due to lower mechanical strength, an acid generation problem upon decomposition, and difficulty in bio-decomposition speed control, application of the foregoing biodegradable polymers was limited, and particularly, due to a polymer characteristic of low mechanical strength, it was difficult to apply the biodegradable polymers to an implant in an orthopedic surgery field or a dental surgery field where it receives a strong load.

In order to overcome such a drawback of a biodegradable polymer, several biodegradable materials were researched, and a typical biodegradable material is a ceramic such as tri-calcium phosphate (TCP) or a composite material of a biodegradable polymer and biodegradable hydroxyapatite (HA).

However, mechanical characteristics of such a material are not remarkably different from that of a biodegradable polymer, and particularly, weak impact resistance of a ceramic material was presented as a fatal drawback as a bio-material. Further, control of biodegradability was not still clearly proved and thus doubt arose in terms of effectiveness.

Further, in order to overcome such a problem, research on a biodegradable metal material is being performed, and a typical biodegradable metal material is magnesium. Because a magnesium material has higher strength than that of a polymer material, a decomposition operation is relatively clearly performed, and the magnesium material is relatively stronger to an impact than a ceramic material, it is expected that the magnesium material will be widely used, but due to still having a fast decomposition speed and a hexagonal structure, there is a drawback that the magnesium material has low elongation and is thus weak on processing.

Because magnesium alloys that are developed to improve this are developed for industry, the magnesium alloys generally include an element in which adaptation is not proved within a living body, such as aluminum and rare-earth metals, and when using them for a living body in consideration that the magnesium alloys are decomposed within a living body, the magnesium alloys are still problematic.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a biodegradable Mg-based alloy and implant having advantages of having biodegradability, easily controlling bio-decomposition speed, and having excellent elongation characteristics and easy workability as well as strength.

An exemplary embodiment of the present invention provides a biodegradable Mg-based alloy that is represented with a composition equation Mg_{100-a-b-c}ZnₐLi_{b}Zr_{c}, wherein a, b, and c of the composition equation are wt % of Zn, Li, and Zr, respectively, and satisfy 0<a≦5, 1≦b≦3, and 0≦c≦1, respectively.

Tensile strength of the biodegradable Mg-based alloy may be 200 MPa or more, and elongation of the biodegradable Mg-based alloy may be 10% or more.

Another embodiment of the present invention provides a biodegradable implant that is represented with a composition equation Mg_{100-a-b-c}ZnₐLi_{b}Zr_{c}, wherein a, b, and c of the composition equation are wt % of Zn, Li, and Zr, respectively, and satisfy 0<a≦5, 1≦b≦3, and 0≦c≦1, respectively.

According to an exemplary embodiment of the present invention, strength of a biodegradable Mg-based alloy can be improved to twice or more than that of a biodegradable polymer, elongation thereof can be improved by 20% or more, and steel processing can be easily performed due to an increase of elongation, while a biodegradable implant may produced as a tube type as well as an implant for orthopedic surgery and dental surgery, and the biodegradable Mg-based alloy can be used as a vein-based biodegradable stent and a non-vein-based biodegradable stent.

### BRIEF DESCRIPTION OF THE DRAWINGS

FIG. 1 is a cross-sectional picture of alloy casting materials Mg3Zn, Mg3Zn0.1Zr, Mg3Zn0.3Zr, and Mg3Zn0.5Zr according to an exemplary embodiment of the present invention.

FIG. 2 is a cross-sectional picture of alloy extrusion materials Mg3Zn, Mg3Zn0.1Zr, Mg3Zn0.3Zr, and Mg3Zn0.5Zr according to an exemplary embodiment of the present invention.

FIG. 3 is a cross-sectional picture of alloy casting materials Mg3Zn, Mg3Zn0.5Li, Mg3Zn1Li, Mg3Zn3Li, Mg3Zn5Li, and Mg3Zn7Li according to an exemplary embodiment of the present invention.

FIG. 4 is a cross-sectional picture of alloy extrusion materials Mg3Zn, Mg3Zn0.5Li, Mg3Zn1Li, Mg3Zn3Li, Mg3Zn5Li, and Mg3Zn7Li according to an exemplary embodiment of the present invention.

FIG. 5 is a cross-sectional picture of alloy casting materials Mg3Zn1 Li0.3Zr, Mg3Zn1Li0.5Zr, Mg3Zn5Li0.3Zr, and Mg3Zn5Li0.5Zr in which 0.3Zr and 0.5Zr are added to alloys Mg3Zn1Li and Mg3Zn5Li, respectively according to an exemplary embodiment of the present invention.

FIG. 6 is a cross-section picture of alloy extrusion materials Mg3Zn1 Li0.3Zr, Mg3Zn1Li0.5Zr, Mg3Zn5Li0.3Zr, and Mg3Zn5Li0.5Zr in which 0.3Zr and 0.5Zr are added to alloys Mg3Zn1Li and Mg3Zn5Li, respectively according to an exemplary embodiment of the present invention.

FIG. 7 is a tensile strength experiment result of an Mg alloy extrusion material that is produced according to an exemplary embodiment of the present invention.

FIG. 8 illustrates a hydrogen generation speed through a dipping experiment of Mg and alloy materials Mg3Zn, Mg3Zn0.1Zr, Mg3Zn0.3Zr, and Mg3Zn0.5Zr according to an exemplary embodiment of the present invention.

FIG. 9 illustrates a hydrogen generation speed through a dipping experiment of Mg and alloy materials Mg3Zn, Mg3Zn0.5Li, Mg3Zn1Li, Mg3Zn1Li0.3Zr, and Mg3Zn1Li0.5Zr according to an exemplary embodiment of the present invention.

FIGS. 10A and 10B are a graph illustrating cell toxicity of a magnesium alloy that is produced according to an exemplary embodiment of the present invention.

FIG. 11 is a ternary phase diagram of an alloy Mg—Zn—Li at 300° C.

### DETAILED DESCRIPTION OF THE EMBODIMENTS

These and other objects of the present application will become more readily apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this

### DETAILED DESCRIPTION

In order to achieve the object, an exemplary embodiment according to the present invention provides a biodegradable Mg-based alloy having strength and ductility appropriate to a living body.

That is, an exemplary embodiment according to the present invention relates to a biodegradable Mg-based alloy and implant in which a bio-decomposition speed can be easily controlled and having excellent strength and elongation, and the biodegradable Mg-based alloy and implant are represented with a general equation Ma wherein a, b, and c of the composition equation are wt % of Zn, Li, and Zr and satisfy 0<a≦5, 1≦b≦3, and 0≦c≦1, respectively. In an exemplary embodiment according to the present invention, Zn and Li are essential constituent elements, and in order to improve elongation, Li is added, but when Li is excessively added, it may be inappropriate to a living body and thus Li is limited to 3 wt % or less.

In an exemplary embodiment according to the present invention, a biodegradable implant is an artificial material that is decomposed within the body to be absorbed or exhausted when healing is complete after a predetermined period has elapsed after a surgical operation, of which a secondary surgical operation for removing the implant is not required, and of which the biodegradable implant can preemptively prevent a side effect such as inflammation or foreign material reaction that may be caused when the implant remains within the body. For example, a biodegradable implant material is a material that is widely defined to include a material of a cardiovascular stent as well as a fixed plate and a screw using in orthopedic surgery, dental surgery, plastic surgery, and maxillofacial surgery.

In an exemplary embodiment according to the present invention, an Mg_{100-a-b-c}ZnₐLi_{b}Zr_{c} composition basically includes a quantity of a level having non-harmfulness within a human body due to dissolving of a constituent element by adjustment of a decomposition speed within a living body, and selects an area in which elongation is greatly improved while having strength of 200 MPa or more. In the composition equation, zinc (Zn) and lithium (Li) are minerals constituting a body and were selected as a constituent element of a very small amount within a living body.

In an exemplary embodiment according to the present invention, when a is 5 or more, strength increases, but the decomposition speed increases and thus an amount of ions that are eluted upon decomposition may have an influence within a living body, and when b is 3 or more, elongation increases, but strength is deteriorated, and ion elution by an increase of a decomposition speed like with zinc (Zn) may have an influence within the living body and thus the ranges of a and b are limited to the above ranges. Further, zirconium (Zr) may improve a property by a microsize of a crystal grain, but when ions are eluted within a living body, a problem may occur and thus in an exemplary embodiment according to the present invention, c is limited to 1 or less. Further, Zr is not an essential constituent component that should be necessarily added, but is added to improve elongation over that of a common alloy, and in an exemplary embodiment according to the present invention, Zr is limited to 1 wt % or less.

The reason for adding zinc (Zn) to magnesium (Mg) in the composition equation is to form a crystal grain in a microsize and to increase strength, and when simultaneously adding zinc (Zn) and zirconium (Zr) to magnesium (Mg), a microsize effect of a crystal grain may be further increased.

Further, impurities that are included in pure Mg such as iron (Fe) or nickel (Ni) largely increase a corrosion speed of magnesium, and when zinc (Zn) is added to magnesium (Mg), corrosion speed of magnesium can be enhanced. (a) to (d) of FIG. 1 are cross-sectional pictures of structures of alloy casting materials Mg3Zn, Mg3Zn0.1Zr, Mg3Zn0.3Zr, and Mg3Zn0.5Zr according to an exemplary embodiment of the present invention, and when zinc (Zn) and zirconium (Zr) at 3 wt % were added to magnesium (Mg), it can be seen that a crystal grain is formed in a microsize (see (c) and (d) of FIG. 1).

Further, when lithium (Li) is added to magnesium (Mg), elongation may be increased, but when a large amount of lithium (Li) is added to magnesium (Mg), strength may be reduced. Therefore, in order to use magnesium (Mg) as a biodegradable metal, it is important to select and add an appropriate amount of lithium (Li).

Hereinafter, a method of manufacturing a biodegradable Mg-based alloy according to an exemplary embodiment of the present invention will be described.

Because magnesium is generally ignited at a relatively low temperature of about 450° C., a special treatment is necessary when melting it, and thus when manufacturing a magnesium alloy, a very small amount (10 ppm or less) of Be is added to the magnesium alloy, and a fusion material surface is covered using a mixed gas of SF₆, CO₂, and dry air. In this way, an elaborate mixed membrane that is formed with MgNₓ, BeO, MgO, MgF₂, and MgS is formed at the fusion material surface, and thus a magnesium alloy fusion material reaction with oxygen is prevented and stable work can be thus performed.

However, when mixing of impurities should be prudently performed like a bio-material, an oxide forming element such as Be cannot be added to a magnesium alloy and thus it is preferable to melt the magnesium alloy in an inert gas atmosphere such as argon (Ar) that does not react with the magnesium alloy. In order to melt a magnesium alloy, various methods such as a resistance heating method of heating by applying electricity to a resistor, a method of guide heating by flowing a current to a guide coil, or a method using laser or focusing light may be used, but the resistance heating method is the most economical. It is preferable to agitate a melting alloy (fusion material) to well mix constituent elements when melting a magnesium alloy.

For controlling mechanical properties and decomposition speed of the melted magnesium alloy by processing, a method that is known in the art of the present invention may be used. For example, the melted alloy may be shaped using an extrusion process. A structure of a magnesium alloy becomes uniform and mechanical performance can be improved by the extrusion. In this case, extrusion of a magnesium alloy is performed in a range of 250-450° C.

FIG. 11 is a ternary phase diagram of an Mg—Zn—Li alloy at 300° C. and referring to FIG. 11, when Zn and Li are each present at about 6 wt % or less, a magnesium alloy with a hexagonal structure HCP may be obtained.

Further, extrusion of a magnesium alloy may be performed while setting a cross-sectional area decrease ratio (hereinafter referred to as an “extrusion ratio”) before and after extrusion to within a range of 10:1 to 30:1. As an extrusion ratio increases, there is a merit that a microstructure of an extrusion material becomes uniform and a defect that is formed upon casting is easily removed, but in this case, capacity of an extrusion apparatus should be increased, and therefore the extrusion ratio is limited to 10:1-30:1.

Further, in a magnesium alloy composition in an exemplary embodiment to of the present invention, elongation increases and workability is enhanced and thus a tube may be produced using an extrusion or drawing construction method that is known in the art of the present invention.

Hereinafter, a structure, a mechanical property, and a decomposition speed according to a biodegradable Mg-based alloy composition that is is produced through an exemplary embodiment of the present invention will be described in detail.

TABLE 1
Alloy composition

| | Alloy composition |
|---|---|
| Exemplary embodiment | Mg₃Zn |
| | Mg₃Zn_{0.1}Zr |
| | Mg₃Zn_{0.3}Zr |
| | Mg₃Zn_{0.5}Zr |
| | Mg₃Zn₁Li |
| | Mg₃Zn₃Li |
| | Mg₃Zn₅Li |
| | Mg₃Zn₇Li |
| | Mg₃Zn_{0.5}Li_{0.3}Zr |
| | Mg₃Zn_{0.5}Li_{0.5}Zr |
| | Mg₃Zn₁Li_{0.3}Zr |
| | Mg₃Zn₁Li_{0.5}Zr |

First, after alloy compositions of Mg—Zn—Li, Mg—Zn—Zr, and Mg—Zn—Li—Zr were mixed as represented in Table 1, a corresponding mixture was melted using a vacuum atmosphere melting method as described above, was extruded at 25:1, and was produced in a rod shape with a diameter of 10 mm. In an exemplary embodiment according to the present invention, the mixture was produced in a rod shape, but it may be produced as an intermediate material of a plate shape, and after the mixture is produced as the intermediate material, by performing lathe or milling processing or by performing pressing forging of a magnesium alloy, the intermediate material may be produced in a shape of a final product.

In a Mg-based biodegradable metal alloy that is produced with the above method, a microstructure after melting and after melting and extrusion was observed using an optical microscope (OM), and size and phase of a crystal grain were analyzed. A change of such a microstructure has a large influence on mechanical properties and a decomposition aspect of an alloy.

(a) to (d) of FIGS. 1 and 2 illustrate alloys Mg3Zn, Mg3Zn0.1Zr, Mg3Zn0.3Zr, and Mg3Zn0.5Zr that are produced with the above-described casting and extrusion methods, that are ground surfaces thereof, and that are observed with an optical microscope. Referring to FIGS. 1 and 2, it may be observed that a small quantity of zirconium (Zr) is added and thus a size of a crystal grain decreases, and it can be seen that the crystal grain size is further formed in a minute size by extrusion.

In an exemplary embodiment according to the present invention, the number that is described in front of an element symbol represents a mass fraction of a corresponding element. For example, the Mg3Zn0.5Zr indicates a magnesium alloy in which Zn is contained at 3% and in which Zn is contained at 0.5%.

(a) to (f) of FIGS. 3 and 4 illustrate alloys Mg3Zn, Mg3Zn0.5Li, Mg3Zn1 Li, Mg3Zn3Li, Mg3Zn5Li, and Mg3Zn7Li that are produced with the above-described casting and extrusion methods, that are ground surfaces thereof, and that are observed with an optical microscope, and (a) to (d) of FIGS. 5 and 6 are optical microphotographs of extrusion materials and alloy casting materials Mg3Zn1Li0.3Zr and Mg3Zn1Li0.5Zr, and Mg3Zn5Li0.3Zr and Mg3Zn5Li0.5Zr, in which 0.3Zr and 0.5Zr are added to alloys Mg3Zn1Li and Mg3Zn5Li, respectively. In all alloys in which lithium (Li) or lithium (Li) and zirconium (Zr) are simultaneously added, it can be observed that a crystal grain is reduced, and particularly, when lithium is added at 5%, it may be determined that a structure of the alloy is changed, and this is because magnesium of a body centered cubic structure (BCC) is formed due to addition of lithium (Li).

Further, strength of a magnesium alloy material that is produced in an exemplary embodiment was measured in a tensile mode based on ASTM B557. A specimen was fixed to a grip portion of a universal material tester with a force of 8 MPa, and an alignment state thereof was determined. In this case, a strain was measured by an extensometer, and a load speed was 1 mm/min.

FIG. 7 is a graph illustrating mechanical properties of a magnesium alloy sample that is produced by an exemplary embodiment of the present invention, and referring to FIG. 7, tensile strength of 200 MPa was secured in all specimens, it can be seen that elongation is 10% or more, and when lithium (Li) is added, it can be seen that elongation greatly increases. This is because when lithium (Li) is added, the added lithium (Li) has an influence on a crystal structure and a configuration phase of magnesium (Mg) and increases a slip system.

Further, a dipping experiment was performed in a Hanks' solution having a composition of Table 2. The temperature was maintained to 37° C. using a water booth, and a specimen was ground with 2000 grit SiC paper, washed with ultrasonic waves in acetone, prepared by drying in air, and a hydrogen generating amount was measured as a generating amount in an area per unit time.

TABLE 2
Composition of corrosive liquid used for corrosion
test (based on 1 liter entire capacity)

| Component name | Weight (g) |
|---|---|
| NaCl | 8 |
| KCl | 0.4 |
| NaHCO₃ (Sodium Hydrogen Carbonate) | 0.35 |
| NaH₂PO₄•H₂O (A430846 420) | 0.25 |
| Na₂HPO₄•2H₂O (K32618380 408) | 0.06 |
| MgCl₂ | 0.19 |
| MgSO₄•7H₂O (Magnesium Sulfate Heptahydrate) | 0.06 |
| Glucose | 1 |
| CaCl₂•2H₂O (Calcium Chloride Dihydrate) | 0.19 |

FIGS. 8 and 9 are graphs representing a hydrogen generating amount through a dipping test of a specimen that is produced by an exemplary embodiment according to the present invention, and it can be seen that the decomposition speed was reduced due to addition of zirconium (Zr), and when adding lithium (Li) at a very small amount, the decomposition speed was reduced, but as the addition amount of lithium (Li) increased, the decomposition speed increased. Therefore, an alloy composition should be selected in consideration of strength and a decomposition speed.

A cell toxicity test of a specimen was performed based on ISO10993. A cell line MG63 of osteoblasts was cultivated within a CO₂ incubator filled with 5% CO₂ and 95% air at 37° C. using Dulbecco's modified Eagle medium (DMEM: Welgene) to which 10% fetal bovine serum (FBS: Welgene) and 1% antibiotics were added.

Further, a cell toxicity test was performed with an elution liquid that was eluted for 1 day, 4 days, and 7 days with a surface area elution ratio of 0.8 cm²/ml using minimal essential medium (MEM: Welgene) in which FBS was not contained, within a CO₂ incubator that was filled with 5% CO₂ and 95% air at 37° C.

5×10³ cells/ml per well were inoculated into a 96-well cell culture plate and cultivated for 24 hours. After 24 hours, elution liquids for 1 day, 3 days, and 7 days were divided at 100 μl to each well and were maintained for 24 hours in a CO₂ incubator in which 5% CO₂ was contained, and in order to determine a cell survival ratio, CCK-8, which is a cell counting kit, was used. A negative control used a DMEM cell badge, and as a positive control, 0.64% phenol was added to a DMEM cell badge and was used.

FIGS. 10A and 10B illustrate a cell survival rate in a cell MG63 and a cell L929 of a magnesium alloy that is produced in an exemplary embodiment according to the present invention. In most cases, as a time passes, a decrease in survival rate was observed, but in most cases, by showing a cell survival rate of 80% or more, it was determined that little cell toxicity existed.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A biodegradable Mg-based alloy that is represented with a composition equation Mg_{100-a-b-c}ZnₐLi_{b}Zr_{c}, wherein a, b, and c of the composition equation are wt % of Zn, Li, and Zr, respectively, and satisfy 0<a≦5, 1≦b≦3, and 0≦c≦1, respectively.

2. The biodegradable Mg-based alloy of claim 1, wherein tensile strength of the biodegradable Mg-based alloy is 200 MPa or more.

3. The biodegradable Mg-based alloy of claim 1, wherein elongation of the biodegradable Mg-based alloy is 10% or more.

4. A biodegradable implant that is represented with a composition equation Mg_{100-a-b-c}ZnₐLi_{b}Zr_{c}, wherein a, b, and c of the composition equation are wt % of Zn, Li, and Zr, respectively, and satisfy 0<a≦5, 1≦b≦3, and 0≦c≦1, respectively.
